(19) United States
(12) Patent Application Publication
(10) Pub. No.: US 2019/0362829 A1
(43) Pub. Date: Nov. 28, 2019

(54) Optical Prescription Management and Fulfillment System

(71) Applicant: Click Vision Group, LLC., Dallas, TX (US)

(72) Inventor: Brent Simpson, Dallas, TX (US)

(21) Appl. No.: 16/424,345

(22) Filed: May 28, 2019

Related U.S. Application Data
(60) Provisional application No. 62/676,751, filed on May 25, 2018.

(51) Int. Cl. G16H 20/10; G16H 10/60

(52) U.S. Cl. CPC G16H 20/10; G16H 10/60

(57) ABSTRACT

In various implementations, an optical management and/or fulfillment system may retrieve patient prescriptions (e.g., from third party records management systems). The optical management and/or fulfillment system may contact patients regarding optical prescriptions. The optical management and/or fulfillment system may fulfill optical prescriptions and/or confirm optical prescriptions (e.g., for third party fulfillment). The optical management and/or fulfillment system may send notifications to individual(s), doctors associated with the individual(s), and/or third party records management systems.

### CROSS-REFERENCE TO RELATED APPLICATIONS

This application claims the benefit of U.S. Provisional Patent Application No. 62/676,751 entitled “Optical Prescription Management and Fulfillment System” filed on May 25, 2018, which is incorporated by reference herein for all purposes.

### TECHNICAL FIELD

The present invention relates to management and fulfillment of optical prescriptions.

### BACKGROUND

Currently, when individuals visit ophthalmologists and optometrists, they may be prescribed a new optical prescription but not order new glasses or contact lenses. This may result in decreased eye health (e.g., through use of old prescriptions and/or old contact lenses while waiting for new contact lenses via a third party fulfillment service), and/or decrease in patient satisfaction (e.g., since it may be a hassle to order glasses and/or contacts through third parties since the individual may have to upload and/or wait for confirmation of the prescription; and/or since the user may forget to order contacts and run out of contacts). This may result in lost revenue for the doctor's office and may increase management costs for the doctor's office (e.g., since the office may need to confirm the optical prescription to an third party fulfillment service and/or prepare a new paper copy of the optical prescription for the individual).

### SUMMARY

Optical product retailers (e.g., Third Party Fulfillment Services, such as contact lens retailers and/or glasses retailers) are required to verify spectacle and contact lens prescriptions prior to delivering ordered optical product(s) to patient. The verification requirement is a burden to optical practices and optical product retailers, who must verify the prescriptions. In addition, the verification process may cause a substantial delay to patients waiting for ordered optical products until the prescription is verified (e.g., which may cause patients harm as the run out of contact lens, extend the use of old contact lens, manage with older prescriptions, etc.). By pre-verifying optical prescriptions, verification can be provided immediately, which may save providers, patients, and/or retailers time and/or money. The optical prescriptions may be verified prior to receiving the orders for optical products from patients and/or a preverified prescription may be provided to optical product retailers (e.g., in response to a verification request and/or after a product has been requested by a patient).

In various implementations, an optical management and/or fulfillment system may retrieve patient prescriptions (e.g., from third party records management systems). The optical management and/or fulfillment system may contact patients regarding optical prescriptions. The optical management and/or fulfillment system may fulfill optical prescriptions and/or confirm optical prescriptions (e.g., for third party fulfillment). The optical management and/or fulfillment system may send notifications to individual(s), doctors associated with the individual(s), and/or third party records management systems.

In various implementations, a method of provided preverifying optical prescriptions (e.g., for fulfillment of a plurality of optical prescriptions) may be provided. Patient information for a plurality of patients and associated optical prescriptions information may be retrieved from a third party patient management system. A determination may be made whether retrieved optical prescriptions are valid (e.g., based at least partially on optical prescription information in the optical prescription(s)). Identifier(s) may be assigned to optical prescription(s) (e.g., unique identifiers). Identifier information, such as but not limited to, at least a portion of the retrieved patient information and/or the retrieved associated optical prescription, may be associated with identifiers. The assigned unique identifiers and/or the associated identifier information may be stored (e.g., in a memory of the system). A first prevalidated optical prescription may be transmitted (e.g., to a Third Party Fulfillment Service, such as an optical product seller) in response to receiving, from a Third Party Fulfillment Service, a valid first identifier. In some implementations, prior to transmitting a first prevalidated optical prescription, a determination may be made whether the first identifier is valid by determining whether the first identifier comprises one of the stored assigned unique identifiers. The first prevalidated optical prescription may be retrieved based on the first identifier and/or associated identifier information.

In various implementations, a method of verifying optical prescriptions for fulfillment of a plurality of optical prescriptions may be provided. Patient information for a plurality of patients and associated optical prescriptions information may be retrieved from a third party patient management system. A determination may be made whether optical prescription information in retrieved optical prescription(s) are valid as retrieved (e.g., based at least partially on optical prescription information in the optical prescription(s)). Identifier(s) may be assigned to valid optical prescription(s) (e.g., unique identifiers). Identifier information, such as but not limited to, at least a portion of the retrieved patient information and/or the retrieved associated optical prescription, may be associated with identifiers. The assigned unique identifiers and/or the associated identifier information may be stored (e.g., in a memory of the system). An identifier may be removed (e.g., deleted and/or invalidated) at an expiration date of the optical prescription associated with the identifier. Optical prescription(s) associated with a patient may be automatically verified upon receipt of a request from a Third Party Fulfillment Services for verification. For example, a first unique identifier may be received from the Third Party Fulfillment Service, and when the first unique identifier is the same as one of the stored assigned unique identifiers and is associated with the one of the optical prescriptions, the optical prescription may be indicated as valid.

In various implementations, a system (e.g., optical system “OP System”) may be provided for verifying optical prescriptions and/or providing preverified optical prescriptions. The system may include a memory storing instructions (e.g., for obtaining and/or verifying prescriptions) and one or more processors capable of executing instruction(s) to perform various operations of the system. The memory may also store retrieved patient information for a plurality of patients, one or more optical prescriptions for one or more of the patients, a plurality of identifiers, associations between at least one of the retrieved patient information, the plurality of patients, the one or more optical prescriptions, the plurality of identifiers, and/or any other appropriate information. An identifier may be associated with one of the optical prescriptions. Operations of the system may include (e.g. performed by one or more processors configured to execute one or more of the instructions) retrieving, for the plurality of patients, the patient information and the associated optical prescriptions information from a third party patient management system. Operations may include determining if at least a portion of each of the optical prescriptions is valid as retrieved and/or assigning a unique identifier for optical prescription(s). A unique identifiers may be associated with identifier information, such as but not limited to, at least a portion of the retrieved patient information, the retrieved associated optical prescription, and/or any other appropriate information. Operations may include storing the assigned unique identifiers and the associated identifier information in the memory. Operations may, in some implementations, include automatically transmitting a first prevalidated optical prescription in response to receiving, from a Third Party Fulfillment Service, a valid first identifier. For example, prior to transmitting a first prevalidated optical prescription, a determination may be made whether the first identifier is valid by determining whether the first identifier comprises one of the stored assigned unique identifiers. A first prevalidated optical prescription may be retrieved based on the first identifier and associated identifier information.

Implementations of the described process(es) and/or system(s) may include one or more of the following features. An identifier may be automatically removed at an expiration date of the optical prescription associated with the identifier. A notification may be transmitted to a patient associated with an identifier and/or the Third Party Fulfillment Service, if a determination is made that the first identifier is not valid. An optical prescription or portions thereof may be automatically adjusted if a determination is made that the optical prescription (e.g., as retrieved) is not valid. The optical prescription or portions thereof may be adjusted based on doctor preferences and/or retrieved optical product information. The adjusted optical prescription may be valid. In some implementations, a patient may be offered an optical product that matches the patient's optical needs. A first patient may be identified, who visited a prescriber within a predetermined period of time, from the retrieved patient information associated with the first patient. A determination may be made whether the first patient purchased a first optical product based on the patient information associated with the first patient retrieved from the third party practice management system, based on previous optical prescription validation requests, and/or based on previously provided preverified optical prescriptions. If a determination is made that the first patient did not purchase the first optical product, a sales offer for the first optical product may be transmitted to the first patient with the preverified optical prescription associated with the first patient. A notification may be transmitted to patient(s) that includes associated identifier(s). In some implementations, automatically verifying one of the optical prescriptions may include receiving a first unique identifier from the Third Party Fulfillment Services and determining whether the first unique identifier is the same as one of the stored assigned unique identifiers. If a determination is made that the first unique identifier is the same as one of the stored assigned unique identifiers, the optical prescription associated with the first unique identifier may be retrieved (e.g., from a memory of the OP System). The retrieved optical prescription may be transmitted to the Third Party Fulfillment Service as a preverified optical prescription. An identifier (e.g., unique identifier) may be automatically removed when later extracted patient information indicates that the optical prescription associated with the identifiers is not valid (e.g., the optical prescription has been replaced, the optical prescription was ill suited for the patient, etc.). In some implementations, a set of optical products that may be purchased by a patient with a specific optical prescription may be identified and utilized by the OP system. For example, a set of optical products that can be validly filled using the optical prescription information of an optical prescriptions may be identified based on doctor based preferences and/or regulations (e.g., substantially similar products identified by the doctor, the manufacturer, industry, and/or the government). The identifier information may include the set of optical products associated with an optical prescription in some implementations and/or the determined set(s) of optical products may be stored in association with the associated one or more unique identifiers. In some implementations, automatically verifying one of the optical prescriptions may include receiving a first identifier and an identification of a first optical product from the Third Party Fulfillment Services, and determining whether the first unique identifier is the same as one of the stored assigned unique identifiers. If a determination is made that the first unique identifier is the same as one of the stored assigned unique identifiers, a determination may be made whether first optical product is in the stored set of optical products associated with the first unique identifier. A notification may be transmitted to the Third Party Fulfillment Service that the first optical product is verified if the determination is made that the first optical products is in the stored set of optical products and the optical prescription is validated. In some implementations, expiration date(s) for one or more of the optical prescriptions may be determined by the OP System. In some implementations, an optical prescription or portion thereof may be automatically adjusted by the OP System, if a determination is made that the optical prescription is not valid as retrieved. The optical prescription or portion thereof may be adjusted based on doctor preferences and/or retrieved optical product information. The adjustment of the optical prescription may cause the adjusted optical prescription to become valid (e.g., fillable by a retailer).

The details of one or more implementations are set forth in the accompanying drawings and the description below. Other features, objects, and advantages of the implementations will be apparent from the description and drawings.

### BRIEF DESCRIPTION OF THE DRAWINGS

For a more complete understanding of this disclosure and its features, reference is now made to the following description, taken in conjunction with the accompanying drawings, in which:

FIG. 1 illustrates an implementation of an example system for management and fulfillment of optical prescriptions.

FIG. 2 illustrates an implementation of an example process for managing and fulfilling prescriptions.

FIG. 3A illustrates an implementation of an example process to manage identifiers.

FIG. 3B illustrates an implementation of an example process to automatically verify optical prescriptions.

FIG. 4A illustrates how the example processes, to manage fulfillment and/or prescription, which are illustrated in FIGS. 3B and 3C are joined.

FIG. 4B illustrates an implementation of a first portion of the example process to manage fulfillment and/or prescriptions, illustrated in FIG. 3A.

FIG. 4C illustrates an implementation of a second portion of the example process to manage fulfillment and/or prescriptions, illustrated in FIG. 3A.

FIGS. 5A and 5B illustrate a process to manage verification and/or fulfillment of optical prescriptions.

Like reference symbols in the various drawings indicate like elements.

### DETAILED DESCRIPTION

In various implementations, an optical (OP) system may manage and/or fulfill prescriptions. Use of the OP system may facilitate compliance with optical prescriptions (e.g., inhibit use of old prescriptions, encourage use of new prescriptions, record fill record, etc.), ease optical prescription fulfillment for doctors and individuals, and reduce costs associated with confirmation of prescriptions, increase revenue by promoting fulfillment of new prescriptions, and/or reduce computing demands (e.g., processing and/or storage of optical prescriptions). Thus, the OP system may provide benefits to doctor's offices and patients alike.

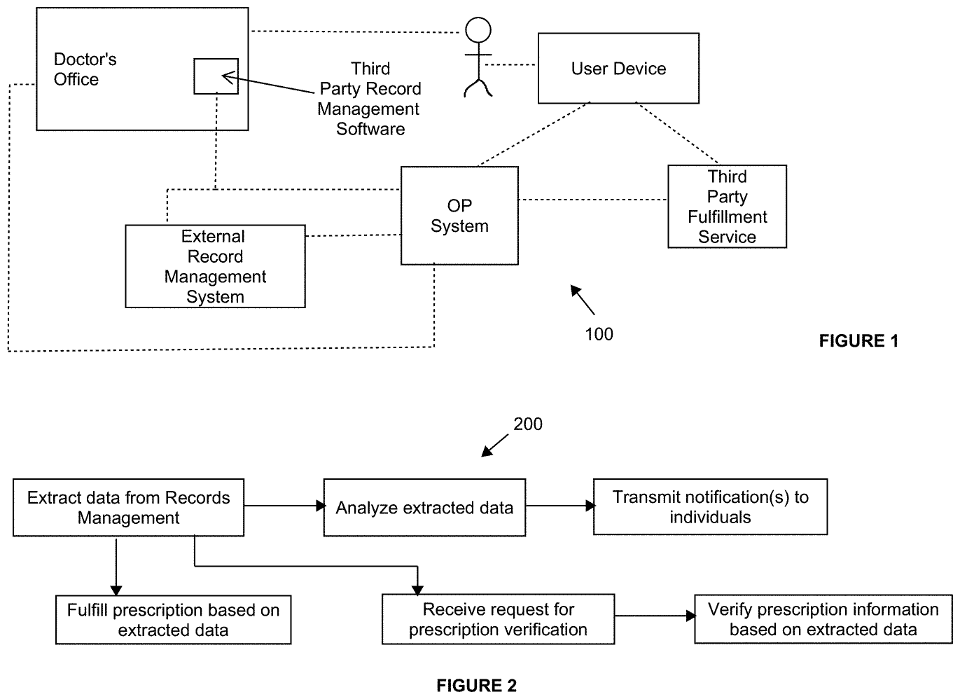

In some implementations, individuals may have a relationship with a doctor's office and the OP system may be utilized based on this relationship. FIG. 1 illustrates an implementation of an OP system based on this relationship. FIG. 2 illustrates a process that may be implemented by systems, such as the described system illustrated in FIG. 1. As illustrated, a person has a relationship with a Doctor's Office (e.g., Ophthalmologist, Optometrist, and/or other licensed optical prescription provider). The Doctor's Office may utilize records management software, such as but not limited to commercially available practice management system (e.g., practice management software and/or electronic records management software). The records management software may reside in part and/or be accessible from the Doctor's Office and may include, in some implementations, an external server, such as the External Records Management System (e.g., cloud based, remote to the Doctor's Office, etc.). The person may explicitly and/or implicitly provide permission for the Doctor's Office to maintain and/or utilize data obtained during visits to the Doctor's Office. The person may extend this permission explicitly and/or implicitly to the OP system (e.g., by agreements with the Doctor's Office, agreements with the OP system, agreements with third party fulfillment services, etc.). Thus, the OP system may comply with requirements and/or best practices for management of patient data.

As previously described, the Doctor's Office may generate data on individuals, with which a relationship exists, through Office visits and/or other types of communication with the individual and/or other parties with a relationship to the individual (e.g., other doctors, hospitals, insurance companies, etc.). For example, notes (e.g., symptoms, diagnosis, past optical product wear satisfaction, etc.) and/or optical prescriptions (e.g, contact lens prescriptions and/or glasses prescriptions) may be generated by the doctor during an office visit. The generated data may be provided to a records management system (e.g., via records management system software). The records management system may store and provide access to the doctor (e.g., for later visits, to look up optical prescription information including current and/or historical optical prescription information).

The data generated and stored by the Doctor's Office may include, but is not limited to, patient name, patient information (e.g., contact address, contact SMS number, contact phone number, emergency contact information, insurance information such as insurance reimbursement information and/or deductible information, etc.), optical prescription (e.g., prescription for glasses, prescription for contact lenses, and/or other ophthalmological prescriptions), and/or any other appropriate information. The optical prescription may include information based on industry and/or government standards such as, but not limited to, power, sphere power, base curve, diameter, cylinder power, axis, prism, add power, color, contact lens brand, recommended coatings for glasses, recommended type of glasses (e.g., progressive, photochromic, etc.), expiration date, prescriber, patient name, optical prescription name, and/or any other appropriate information. Some of the optical prescription information may relate to a specific eye (e.g., right or left) and/or some of the optical prescription information may relate to both eyes, in some implementations. The data generated and/or stored may be in compliance with regulations (e.g., privacy related, transaction related, etc.) and/or best practices. Standardization of data any data provided by a practice is infrequent, the OP System may accommodate variations using various AI tools (e.g., trained on a specific practice records, industry standards, etc.).

The OP system may be in communication with the third party software at the doctor's office and/or via external servers (e.g., External Record Management System). The OP system may extract data associated with one or more individuals. In some implementations, the data associated with one or more individuals may be transmitted (e.g., pushed) from the Records Management System to the OP System. For example, the OP System may include an OP application interface (OP API) that is capable managing communication between the Record Management System (e.g., software residing on a computing device at the Doctor's Office and/or External Record Management System) and the OP API may allow the data to be transmitted (e.g., extracted and/or pushed) to the OP System from the Record Management System. In some implementations, the Doctor's Office may store information locally and/or remotely not in conjunction with a Records Management System. The OP System may be capable of extracting data from the Records Management System and/or data stored in another way by the Doctor's Office (e.g., local memory, scanned files, etc.). Standardization of data any data provided by a practice is infrequent. The OP System may standardize data extracted (e.g., using AI tools, for example, trained on a specific practice's records, and/or multiple practice records).

The OP API may reside at least partially locally (e.g., at the Doctor's Office), may be web-accessible, and/or may reside at least partially at a remote OPI System component. For example, the OP API may reside on a computer at the doctor's office that is capable of accessing third party practice management system. The OP API may include an extraction tool that is capable of extracting data from the Records Management System.

The OP System may receive the extracted information and analyze at least a portion of the extracted information. The OP System may save at least a portion of the extracted information with associations retrieved form with the extracted data (e.g., data may be associated with a patient and/or doctor). In some implementations, the OP System may save optical prescription information and associate one or more portions of the optical prescription information with each other. For example, patient information may be associated with the prescriber (e.g., doctor, optometrist), and a specific optical prescription.

The OP System may analyze the data for marketing purposes (e.g., targeted advertising, discount information, rebate information), fulfillment purposes (e.g., fulfillment of glasses, contact lenses and/or related supplies such as lens solutions and/or glasses lens cleaner), verification purposes (e.g., to verify prescriptions), optical prescription compliance purposes (e.g., fill appropriate number of prescriptions over a period of time), and/or any other appropriate purpose. For example, the OP System may analyze the data to allow secure and compliant automatic verification of optical prescriptions by Third Party Fulfillment Services (e.g., Third Party Fulfillment Services that do not have access to the practice management system).

The OP System may identify optical prescription information and determine if the optical prescription information is valid as written (e.g., complete, contains enough information to be fulfilled, is not contradictory such as by including a base curve that is not available in a particular contact lens brand). If the optical prescription information is incomplete, the OP System may modify the optical prescription (e.g., perfect the optical prescription, provide at least a portion of the missing information). As a non-limiting example, if a contact lens prescription does not include a diameter, but the brand of contact lens identified in the optical prescription includes a single diameter, then the OP System may alter the optical prescription to include the diameter information associated with the prescribed brand of contact lens. As another non-limiting example, if a glasses prescription does not include an expiration date the OP System may automatically insert a one-year or other predetermined time period expiration date (e.g., as provided by the prescriber's preferences and/or state or federal regulations). As another non-limiting example, if a glasses prescription does not include a sphere lens power, the OP System may insert the sphere lens power from an associated contact lens prescription. Another non-limiting example, the optical prescription may include a diameter than is not available in the brand of contact lens prescribed. The OP System may automatically determine the appropriate diameter and perfect the optical prescription, within limits provided by the doctor (e.g., the contact lens brand is only available in 1 diameter size, the previous optical prescription included an appropriate diameter size, the diameter in the optical prescription was close to an available diameter size, the contact lens brand was previous prescribed with an appropriate diameter to the patient, the prescribed time to discard lens is not within the recommended time to discard lens, etc.). In some implementations, the adjustment may be within prescriber provided limits. In some implementations, the OP System may analyze the incomplete optical prescription and determine if the information needed to perfect the optical prescription can be determined by the OP System and may alter the optical prescription to provide the information needed to complete (e.g., perfect) the prescription. The determination of whether the information needed to perfect the optical prescription may be based on other information associated with the individual (e.g., other optical prescriptions, previous records, etc.) and/or information associated with type of glasses or contact lens in the optical prescription (e.g., limitations of availability with certain brands, limitations of options associate with a type of optical prescription, etc.). Information about the type of glasses and/or contact lenses may be retrieved third party sources (e.g., manufacturer website and/or guidelines, regulations, etc.), and/or from memory coupled to the OP System. For example, contact lens manufacturers may include information about manufactured contact lens including types of contact lens, available powers, base curves, colors, length of use, etc.

In some implementations, the adjusted optical prescription may be transmitted to the prescriber to verify and/or approve. The OP System may allow the adjusted optical prescription to be utilized by the OP System unless a notification of rejection is received from the prescriber (e.g., alteration of optical prescription by the prescriber, response to a notification transmitted to prescriber, etc.). In some implementations, the OP System may not allow the adjusted optical prescription to be utilized until the approval is received from the prescriber.

In some implementations, if a determination is made that the OP System can not provide the information for the incomplete optical prescription and/or can provide a portion of the information needed to perfect the prescription, the OP System may transmit a notification to the Doctor's Office. The notification may be via the Records Management System, via automated phone call, via messaging (e.g., SMS, iMessage, etc.), via email, etc. The OP System may request via the notification that the Doctor's Office provide information to perfect the incomplete optical prescription (e.g., via the Records Management System and/or directly to the OP System). The OP System may receive the requested information from the Doctor's Office (e.g., directly from the Doctor's Office and/or via extraction of information from the Records Management System) and reanalyze the optical prescription to determine if the optical prescription is complete. These operations may be continued more than one time to allow a complete optical prescription to be obtained, in some implementations. In some implementations, the OP system may transmit modified optical prescriptions and/or generated prescriptions to the Doctor's office for approval.

In some implementations, the alterations may be allowed unless a rejection notification is received from the Doctor's Office. The modifications may not be allowed unless an approval notification is received from the Doctor's Office in some implementations. The OP System may save the adjusted and complete optical prescription and/or the originally extracted optical prescription. Thus, in some implementations, the OP System may adjust the retrieved optical prescription information that was determined to not be valid (e.g., fillable as written) such that the adjusted optical prescription information is a valid optical prescription information.

Since glasses prescriptions and contact lens prescriptions are discrete (e.g., one optical prescription may not be used by an Third Party Fulfillment Service to provide both or the other optical product). In some implementations, the OP System may automatically generated contact lens prescriptions based on glasses prescriptions when prompted by the Doctor's Office. The OP System may be able to automatically generate the glasses prescription based on the extracted contact lens prescription or vice versa. As a non-limiting example, if a glasses prescription is not provided but a contact lens prescription is provided, a glasses prescription may be automatically generated based on the contact lens prescription and/or adjustments (e.g., industry guidelines for differences, such as in power; doctor optical prescription preferences, etc.). In some implementations, the OP System may make adjustments (e.g., to spherical lens power) based on standard practices and/or doctor provided criteria (e.g., glasses prescriptions may be 0.25 stronger than contact lens prescriptions).

In some implementations, an individual may request contact lenses and/or glasses prescriptions that were not in the extracted information and the OP System may provide the requested optical prescription automatically and/or upon approval of the request by the associated Doctor's Office. An individual may have forgotten to request and/or a doctor may have forgotten to provide one type of optical prescription while providing a different type of optical prescription. For example, patients commonly forgot to request glasses prescription when obtaining a contact lens prescription (e.g., during a contact lens fitting examination). The OP System may automatically generated the requested type of optical prescription based at least partially on at least a portion of the extracted data (e.g., previous prescriptions, current prescriptions, etc.), manufacturer information, standard practices, and/or doctor provided criteria. By allowing the individual to request the optical prescription via the OP System, costs at the Doctor's Office may be decreased.

In some implementations, the OP System may request verification from the prescriber of an optical prescription perfected by or in part by the OP System. For example, the altered optical prescription may be transmitted to the Doctor's Office for confirmation of the accuracy of the portions provided by the OP System. The OP System may allow fulfillment of the altered and perfected optical prescription and/or may inhibit fulfillment until confirmation is received by the OP System.

The OP System may transmit one or more notifications to individuals based on at least a portion of the extracted information. For example, the OP System may transmit marketing notifications, such as discounts and/or rebates for a prescribed brand, glasses fulfillment, contact lens fulfillment, etc.

As another nonlimiting example, the OP System may transmit notifications to individuals to fulfill a prescription (e.g., new and/or existing). In some implementations, the OP System may monitor the fulfillment of an optical prescription (e.g., through the OP System, through Doctor's Offices, and/or via third party fulfillment services) and transmit notifications to an individual based on the monitoring. For example, refill reminders may be transmitted, supply (e.g., contact lens solution, etc.) reminders may be transmitted, fulfillment of a new optical prescription reminder may be transmitted, and/or any other appropriate notification may be transmitted. Individuals commonly find contact lens are expensive and stretch the use of a pair of contact lenses; however, use of contact lens longer than the prescribed duration may cause damage to eyes (e.g., infections, scratches, etc.). By monitoring the fulfillment of the optical prescriptions, the OP System may be able to improve patient health by monitoring contact lens purchasing and providing notifications to remind patients to comply with prescribed durations (e.g., notifications to purchase and/or change lenses). The notifications may be via message (e.g., SMS), email, automated phone call, and/or any other appropriate type of message. The OP System may identify the way to transmit the notification based on the extracted information (e.g., contact information provided in the Records Management System). Since the OP System may be able to monitor more than one Third Party Fulfillment Service (e.g., doctor's office and/or third party sellers), the OP System may be able to more accurately monitor compliance with prescribed durations of use for contact lenses and thus improve patient health and/or decrease the likelihood that a patient will ignore a notification regarding refill and/or purchase (e.g., since the information may be more accurate and/or since the notification may remind the patient to repurchase from the individual's preferred Third Party Fulfillment Service).

The notifications transmitted by the OP System may provide a way to fulfill the prescription. For example, the notification may include a link to a website to fulfill the optical prescription. In some implementations, the OP System may transmit an identifier (e.g., alphanumeric, image, bar code, QR code, etc.) that may be provided to a website (e.g., to allow fulfillment, order history, presentation of at least a portion of the extracted information, presentation of marketing notifications, etc.). The identifier may be associated with the optical prescription and allow automatic verification of the optical prescription. The notification may allow a response to be provided (e.g., text back to the OP System). The notification (e.g., an email) may include a button or field that allows a user to select a response.

In some implementations, an individual may be prompted to provide consent to one or more notifications and/or access to information associated with the individual. For example, the OP System may transmit a notification to the individual to consent and/or confirm consent to the access provided by the relationship of the individual to the Doctor's Office.

In some implementations, the OP System may transmit the optical prescription to the individual (e.g., as extracted and/or as perfected at least partially by the OP System). The OP System may transmit a notification, to the individual (e.g., a user device of the individual) that includes whether to fulfill the optical prescription. A user may then transmit a request to fulfill the optical prescription to the OP System and the OP system may coordinate fulfillment of the optical prescription (e.g., via the OP System and/or via third party fulfillment services). In some implementations, an individual's information such as, but not limited to, payment information and/or shipping address may be included in the extracted information and the individual may utilize and/or approve use of the individual's information for fulfillment.

The OP System may identify and transmit a notification to a user fulfilling a prescription regarding whether the individual would like to purchase one or more other related products and/or services, such as but not limited to contact lens cases, contact lens cleaners, saline drops, glasses cleaner, glasses cases, glasses chains, clip on sunglasses for glasses, sport bands for glasses, etc. The individual may request fulfillment of one or more of the additional related products based on the notification by the OP System. The OP System may coordinate fulfillment of the additional related products and/or services (e.g., via the OP System and/or via third party fulfillment services).

In some implementations, the OP System may determine, based at least partially on extracted information and/or requests for verification of the optical prescription (e.g., in the extracted information and/or from third party fulfillment services), whether the individual has already fulfilled the optical prescription (e.g., for the full optical prescription length, such as a year supply of contacts; and/or for a period of time), and may not transmit the notification regarding fulfillment of the optical prescription based on this determination. In some implementations, the OP system may transmit a notification regarding related products and/or services (e.g., contact lens solution) based on a determination that the optical prescription was fulfilled.

In some implementations, when a determination has been made that an optical prescription has been fulfilled (e.g., via the OP System, Doctor's office, and/or Third Party Fulfillment Services), the OP System may transmit one more reminder notifications. For example, the OP System may transmit a reminder to reorder contact lenses (e.g., based on the quantity ordered in a fulfillment and/or date of fulfillment; based on the expiration of the optical prescription, etc.).

In some implementations, the OP System may monitor fulfillment of optical prescriptions and supplies to facilitate user tracking (e.g., for health care benefit reimbursement, for budgeting, etc.), to determine common related products and/or services purchased with fulfillment of optical prescriptions (e.g., to use in future marketing notifications for the individual or other individuals, who do or do not use similar products), to report to the Doctor's Office, and/or for any other appropriate purpose. For example, the OP System may report fulfillment of optical prescriptions and supplies to the Doctor's office via a notification and/or by entry in the Record Management System (e.g., via the OP API). A doctor reviewing the information may then be able to provide advice and/or recommendations based on proper and/or improper usage (e.g., not enough contacts and/or contact lens solution purchased). For example, the OP System may determine whether the fulfillment of optical prescriptions and/or supplies was compliant based on doctor supplied, default values, and/or best practices and may generate a flag (e.g., compliant, unknown, noncompliant) in the notification and/or Records Management System.

In some implementations, the OP System may generate one or more additional notifications and/or entries in the Record Management System for a Doctor's Office. For example, the OP System may analyze the extracted information for an individual and generate a topic list for a future visit. The topic list may be based on testing performed on the individual, individual's medical history, and/or fulfillment monitoring. For example, the topic list may include diseases such as diabetes, macular degeneration, and/or hygiene. In some implementations, the determination of which topics to include on a topic list may be based on models generated by the OP System based on extracted information over the population and/or portions of the population. The modules may be generated based on studies, doctor provided criteria, and/or default values (e.g., topic list for contact wearers includes reminder to take out at night). In some implementations, progression of an individual's health may be determined from the extracted information to generated models (e.g., 85% chance of glaucoma based on individual health) to predict other patient's health.

In some implementations, the OP System may alter and/or scrub extracted data. For example, if a model is shared with a doctor, then individual patient information may be scrubbed from the model to inhibit identification of the individual and to comply with regulations (e.g., privacy and/or patient rights).

In various implementations, the OP System may operate as an optical prescription information verifier to provide compliance with regulations (e.g., state and/or federal laws). When fulfilling optical prescriptions, fulfillers are required to verify the optical prescription with the associated doctor's office. However, the Doctor's office is not paid for providing the service or is minimally paid for providing the service. Additionally, there is a time delay since the Doctor's Office must pull the records for the individual, verify the optical prescription, and then provide a response (e.g., via fax, website, etc.). Furthermore, received optical prescriptions may include errors and/or incomplete information (e.g., since the information is often provided by a patient or by OCR of an optical prescription), which takes time for the Doctor's Offices to correct, as required. Doctor's Offices are required to provide verification of optical prescriptions by law but since the time spent is not or under reimbursed, optical prescription verification is a cost to the Doctor's Office. Thus, removing the Doctor's Office active participation in the process may reduce costs for the Doctor's Office, increase user satisfaction (e.g., since delays may be minimized), and/or increase fulfillment service provider satisfaction (e.g., since delays may be minimized). Additionally, costs commonly exist for Third Party Fulfillment Services (e.g., third party sellers without access to the practice management system of prescribers) to verify optical prescriptions, as required. Commonly, Third Party Fulfillment Services may pay approximately $5 per optical prescription for verification, which quickly multiplies on low volume orders and/or over hundreds of thousands of orders fulfilled. Thus, providing an automatic optical prescription verification via the OP System may decrease costs for the Third Party Fulfillment Services.

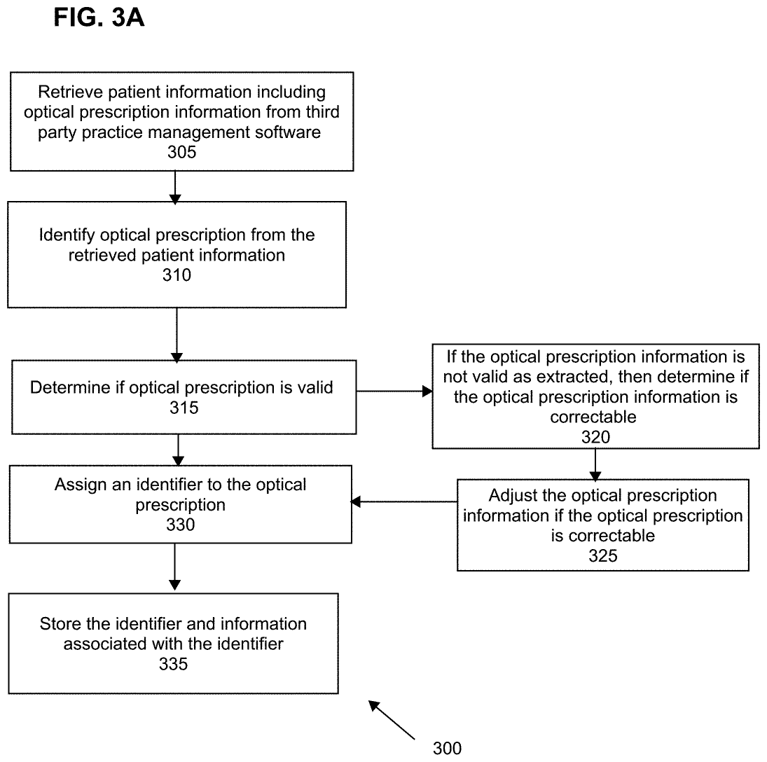

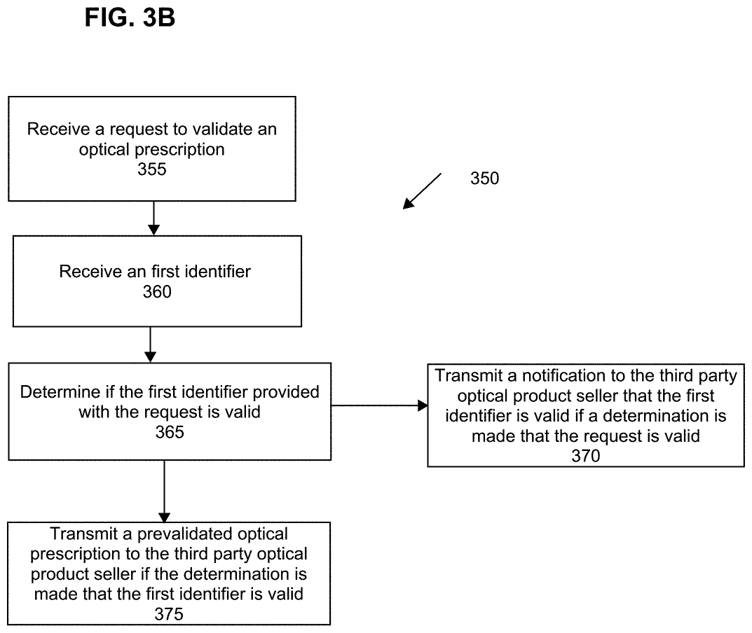

FIG. 3A illustrates an implementation of a process 300 to facilitate verification of optical prescriptions. The described process may be implemented by the described OP System (e.g., modules of the OP System may be executed by the processor(s) of the OP System to perform one or more of the described operations of the process). Patient information (e.g., including optical prescription) may be retrieved from third party practice management system (operation 305). The patient information may be retrieved for one or more patients associated with one or more prescribers (e.g., doctors). Thus, a plurality of patient information associated with a plurality of patients may be retrieved (e.g., in each retrieval, over a period of time, etc.). The patient information may be retrieved automatically (e.g., by the OP System), semi-automatically, and/or manually in batches (e.g., periodically and/or irregularly) and/or continuously. In some implementations, the prescriber and/or practice management system may upload the patient information to the OP System.

For one or more of the patients, the patient information may be analyzed. The optical prescription information may be identified from the retrieved patient information (operation 310). For example, the patient information may be parsed to identify the information associated with the optical prescription (e.g., type of prescription such as glasses or contact lens; brand; power, etc.).

A determination may be made whether the optical prescription is valid (operation 315). An optical prescription may be valid if it is fillable by an Third Party Fulfillment Service as written (e.g., industry and/or government regulations may indicate the information needed on an optical prescription).

If a determination is made that the optical prescription is not valid, then a determination may be made whether the optical prescription is correctable (operation 320). For example, as previously described, the optical prescription may be missing information and/or include invalid information (e.g., old contact lens brand name, unavailable base curve, etc.). Some of this missing and/or incorrect information may be correctable by the OP System and some may not be correctable (e.g., missing power). The OP System may retrieve information (e.g., from a memory associated with the OP System and/or third parties such as contact lens manufacturers) and use this retrieved information at least partially in determining whether the incorrect and/or missing information in the optical prescription is correctable, in some implementations. In some implementations, commercially available AI tools may be utilized to determine whether the incorrect and/or missing information in the optical prescription is correctable. For example, doctor preferences may be generatable by commercially available AI tools to provide information missing from the optical prescription (e.g., expiration dates, power differences prescribed between glasses and contacts, allowable substitutions of contact lens types such as color for noncolored lens and/or substitution of two week for one month lenses).

If a determination is made that the optical prescription that is not valid is adjustable by the OP System to be valid and/or valid with respect to one or more parts of the optical prescription, then the OP system may adjust the optical prescription. For example, the OP System may provide information from a contact lens prescription to generate and/or complete a glasses prescription. As another nonlimiting example, the OP System may adjust a base curve to a base curve previously provided to the patient and/or available in the contact lens type. As another nonlimiting example, the OP System may add and/or adjust an expiration date. As another nonlimiting example, the OP System may correct a spelling of a contact lens type. As another nonlimiting example, the OP System may replace a no longer available type of contact lens with an available contact lens (e.g., that is similar as determined by commercially available AI tools and/or manufacture information).

In some implementations, a notification regarding the adjusted optical prescription and/or portions thereof (e.g., adjusted portions) may be transmitted to the prescriber for verification, refusal, and/or adjustment. In some implementations, the OP system may allow the adjusted optical prescription to be utilized unless a refusal of the adjustment is received by the OP System from the prescriber (e.g., in response to the notification from the OP System and/or from an updated practice management system record, etc.).

In some implementations, if a determination is made that the optical prescription is not correctable (e.g., in whole and/or in part), a notification may be transmitted to the prescriber requesting additional information to correct the optical prescription and/or correction of the optical prescription (e.g., directly with the OP System and/or in the third party practice management system).

Thus, the OP system may include a plurality of valid optical prescriptions (e.g., as retrieved and/or adjusted optical prescriptions) for a plurality of patients that are associated with a plurality of providers. The OP System extracts the optical prescription and ensures completeness (e.g., via analysis and/or completion via the OP System and/or Doctor's Office); thus, the OP System can then verify prescriptions on behalf of the Doctor's Office. In various implementations, the OP system can independently (e.g., without additional approval from the doctor's office) and automatically verify prescriptions for Third Party Fulfillment Services (e.g., physical stores such as Warby Parker, Walmart, Lens Crafters, other doctor's offices; and/or online sellers such as 1800Contacts, Walmart, Zenni).

The OP System may receive (e.g., directly or indirectly via an agent) a verification request from a Third Party Fulfillment Service (e.g., optical product seller) for verification of an optical prescription, which may include the optical prescription and/or an associated individual's information. In various implementations, the OP system may or may not utilize (e.g., internally and/or externally) an identifier to facilitate verification of optical prescriptions.

In some implementations, the OP System may not utilize identifiers to verify prescription information. The OP System may analyze the received optical prescription based on the complete optical prescription stored in the OP system for the associated individual and determine whether the received optical prescription can be verified. The OP System may then transmit a notification to the Third Party Fulfillment Service that the optical prescription can be verified. The OP System may transmit one or more notifications to the individual (e.g., based on the individual's contact information stored in the OP System) and/or the prescriber. For example, the OP System may transmit a notification that the optical prescription has been verified, transmit a notification with an advertisement, and/or transmit a notification for related products and/or services.

In some implementations, the OP System may utilize identifiers associated with optical prescriptions to facilitate verification of the optical prescription. The OP System may assign an identifier to an optical prescription (operation 330). The OP System may or may not assign the identifier prior to determining if the optical prescription is valid. The identifier may be any appropriate type of identifier (e.g., bar code, QR code, alphanumeric, etc.) and may be associated with the optical prescription and/or portions thereof. For example, the identifier may be associated with a patient, a lens type, a power, prescriber, an expiration date, etc. The identifier may be associated with a single optical prescription in some implementations. Thus, a patient may be associated with more than one identifier.

The identifier and/or information associated with the identifier may be stored (operation 335). The identifier, information associated with the identifier, and/or their associations may be stored in a memory accessible by the OP System (e.g., local and/or remote repository).

In some implementations, a determination may be made of a set of products allowed by the optical prescription. Based on doctor preferences (e.g., determined by preferences provided by a doctor and/or learned by commercially available AI tools regarding extracted optical prescriptions) a set of products may be allowed with a specific optical prescription. For example, similar noncolored lenses may be allowed by a specific doctor when colored lenses are prescribed. As another example, glare reducing lenses may be prescribed, but glasses without glare reducing coatings may be allowed without a new prescription. As another nonlimiting example, lenses without coatings may be prescribed, but glasses with one or more coatings (e.g., scratch resistance, computer lenses, etc.) may be allowed without a new prescription. In some implementations, a range of products may be classified as similar (e.g., by the manufacturer and/or the industry) and thus the products may be substituted for each other without obtaining an additional prescription. The set of optical products allowed may be stored in association with an identifier to allow verification of optical products in the set of optical products.

In some implementations, the OP system may at least partially automatically manage the identifiers and/or optical prescriptions. For example, identifiers may be deleted and/or indicated as invalid after an expiration date. As another example, identifiers may be deleted and/or indicated as invalid based on additional information received from the practice management system associated with a provider (e.g., office visits notes lenses were ill fitting and/or replaced with a different prescription). By automatically managing the identifiers, the risk of optical product purchases that are incorrect and/or allowed after the expiration date may be reduced (e.g., when compared with individual's having to provide a prescription).

In various implementations, the identifier may be provided to providers, patients, and/or Third Party Fulfillment Services. For example, the OP System may provide the identifier to the patient such that the patient may provide the identifier to a patient selected Third Party Fulfillment Service. The provided identifier may be utilized by the Third Party Fulfillment Service to obtain automatic verification of the optical prescription for the patient. The identifier may be utilized in place of providing prescription information (e.g., from the patient to a seller) and/or to allow automatic verification.

The OP System may receive a request from a Third Party Fulfillment Service for verification of an optical prescription and/or verification of an optical product purchase. FIG. 3B illustrates an implementation of an example process 350 for automatically verifying optical prescriptions using an identifier. An individual may receive (e.g., from a prescriber and/or OP System user interface and/or notification) a first identifier. The first identifier may be associated with the individual and an optical prescription of the individual. A request may be received from a Third Party Fulfillment Service to validate an optical prescription (operation 355). The first identifier may be received (operation 360). For example, the individual may provide the first identifier to the Third Party Fulfillment Service when ordering an optical product. The Third Party Fulfillment Service may transmit the identifier to the OP System for verification. A determination may be made whether the first identifier is valid (operation 370). The OP System may determine if the first identifier is valid based on whether a similar first identifier is stored in a memory of the OP System (e.g., when the OP System automatically deletes invalid identifiers) and/or based on an expiration date associated with the identifier.

In some implementations, a notification may be transmitted that the first identifier is valid if a determination is made (e.g., by the OP System) that the first identifier is valid (operation 370). In some implementations, the prescription information may be encoded in the first identifier and the Third Party Fulfillment Service may decode (e.g., based on information provided by the OP system) the first identifier to retrieve the optical prescription associated with the first identifier. For example, a QR code may include the optical prescription. As another nonlimiting example, the first identifier may be transmitted to the individual with the prescription information (e.g., in a substantially nonmodifiable format), and the individual may transmit the first identifier and the optical prescription information to the Third Party Fulfillment Service. Once the first identifier is validated by the OP System, the Third Party Fulfillment Service may utilize the optical prescription information to provide optical products in the optical prescription to the individual.

In some implementations, if a determination is made that the first identifier is valid, a prevalidated optical prescription may be transmitted to the third party product seller that requested validation of an optical prescription (operation 375). Since the optical prescription of the OP System is from information extracted from the provider's practice information, the OP System can transmit the prescription to the Third Party Fulfillment Service such that it complies with validation requirements (e.g., the optical prescription is prevalidated).

In some implementations, the third party seller may request validation of a specified optical product (e.g., type, power, etc.). For example, an individual may transmit a first identifier to a Third Party Fulfillment Service along with a request to purchase a specified optical product. The individual may or may not provide optical prescription information to the Third Party Fulfillment Service. The request for validation of the specified optical product for an individual may be received (e.g., by the OP System from the Third Party Fulfillment Service). Information about the specified optical product may be identified (e.g., by the OP System), such as kind (e.g., glasses or contacts), type of contacts, brand of contacts, power, sphere, cylinder, axis, and/or any other appropriate type of information. Information about the individual may be received (e.g., patient name, patient address, prescriber, etc.). The identifier associated with the received patient information may be retrieved. The preverified optical prescription (e.g., the optical prescription previously determined by the OP System to be valid and/or fillable) may be retrieved and/or a set of products associated with the preverified optical prescription may be retrieved. A determination may be made whether the specified optical product (e.g., kind, brand, type, power, etc.) can be validated based on the preverified optical prescription and/or set of products associated with the preverified optical prescription. The specified optical product may be validated for the individual if the determination is made that the specified optical product is allowed by the preverified optical prescription and/or set of products associated with the preverified optical prescription (e.g., the prescribed kind, power, etc. is the same; the product requested is in the set of products associated with the preverified optical prescription). The specified optical product may be not be validated for the individual if the determination is made that the specified optical product is not allowed by the preverified optical prescription and/or set of products associated with the preverified optical prescription. In some implementations, the specified optical product information may be modified and transmitted to the Third Party Fulfillment Service to allow the optical product to be verified (e.g., prescription may be adjusted, brand may be adjusted, etc.).

In various implementations, the OP System may determine that the optical prescription can not be verified (e.g., due to missing information, expiration, wrong information). The OP System may, in some implementations, provide a completed optical prescription that can be verified and is associated with the individual to the Third Party Fulfillment Service (e.g., optical product seller).

In various implementations, the OP System may transmit a notification to the Doctor's Office and/or enter into the Records Management System of a Doctor's office, a notification based on the fulfillment by the Third Party Fulfillment Service.

In various implementations, a Doctor's Office may be able to access monitored information for an individual via an interface generated by the OP System. For example, the Doctor's office may be able to access order records.

In various implementations, optical prescriptions and/or optical product orders may be verified and/or preverified optical prescriptions may be provided by the OP System (e.g., for fulfillment of a plurality of optical prescriptions). Patient information for a plurality of patients and associated optical prescriptions information may be retrieved from a third party patient management system. For example, rather than just being based on a single provider and/or a single third party patient management system, the OP System could provide verifications (e.g., of optical prescriptions and/or optical product orders; preverified optical prescriptions) for a plurality of patients associated with a plurality of providers. The patient information may include any appropriate information, such as patient name, address, insurance, history, etc. Optical prescriptions may include any type of optical prescription (e.g., spectacle or “glasses” prescriptions and/or contact lens prescriptions). The optical prescription information may include information required by industry and/or regulatory standards and/or information required to fill an optical prescription. The data in patient information and/or optical prescription may not be stored in the third party practice management system in a standardized form. Thus, in some implementations, the OP system may standardize at least a portion of the information retrieved from the third party patient management system (e.g., using commercially available AI tools that learn standardization via prescriber records, other prescriber records, commonly used terminology, other appropriate learning tools, or combinations thereof).

A determination may be made whether retrieved optical prescriptions are valid (e.g., based at least partially on optical prescription information in the optical prescription(s)). An optical prescription may be considered valid (e.g., as retrieved) if the optical prescription is fillable by a retailer with the information provided and/or complies with industry and/or government regulations. For example, if an optical prescription did not include a lens power (e.g., sphere and/or cylinder), an expiration date, a patient name, etc.

In some implementations, if a retrieved optical prescription is not valid as retrieved, a notification may be provided to the prescriber (e.g., email, text, message in third party practice management system, etc.). The prescriber may provide corrected optical prescription information directly to the OP system, in response to the notification, and/or via the third party practice management system.

In some implementations, if a retrieved optical prescription is not valid as retrieved, the OP System may determine whether the retrieved optical prescription is correctable. If the OP System determines that the retrieved optical prescription is not correctable (e.g., contact lens type for a new contact lens wearer) and/or partially correctable then the OP system may transmit a notification to the provider. For example, a prescriber may provide a listing of which types of information (e.g., expiration date, base curve, etc.) and/or in which patients (e.g., long time contact wearers vs new contact wearers, glasses wearers, etc.) may be automatically adjusted in an optical prescription. In some implementations, adjustments may be restricted in specified portions of an optical prescription. For example, government regulations may inhibit automatic adjustments in portions of the optical prescription and/or industry regulations may inhibit automatic adjustment certain fields, such as of expiration dates to longer than a predetermined period. If an OP system determines that the invalid optical prescription is correctable and/or partially correctable, the OP System may modify the optical prescription to create an adjusted optical prescription. For example, the OP System may utilize manufacturer information (e.g., to provide information regarding ranges of options in available products, substitute products, etc.), prescriber information, and/or any other appropriate information. The OP system may utilize commercially available AI tools to create a platform that learns which parts are correctable and how to correct these parts (e.g., unavailable base curve in specified contact lens as retrieved, missing contact lens brand in long time contact wearer). For example the AI tools may be trained and learn based on previous corrections of optical prescriptions (e.g., provided by prescriber or other prescribers). The adjusted optical prescription may be valid (e.g., fillable as written in the adjusted optical prescription).

A prescriber may or may not review and/or approve the adjusted optical prescription prior to allow the OP system to utilize the adjusted optical prescription in verifications. In some implementations, the OP system may be inhibited from utilizing adjusted optical prescriptions or a subset of adjusted optical prescriptions (e.g., due to the nature of the adjustment) until a prescriber associated with the original optical prescription and/or another prescriber (e.g., a prescriber tasked with approvals and rejections) approves the adjusted optical prescription. In some implementations, the prescriber may reject an adjusted optical prescription and/or provide an adjustment to the optical prescription in response to the review.

Identifier(s) may be assigned to optical prescription(s) (e.g., unique identifiers). In some implementations, identifiers may be assigned to valid optical prescriptions (e.g., valid as retrieved, valid as corrected by a provider and/or valid as adjusted by the OP system). In some implementations, assignment of an identifier may be inhibited until an optical prescription (e.g., as retrieved and/or adjusted) is determined to be valid. Inhibiting assignment of an identifier may reduce erroneous validations. Identifier information, such as but not limited to, at least a portion of the retrieved patient information and/or the retrieved associated optical prescription, may be associated with identifiers. The assigned unique identifiers and/or the associated identifier information may be stored (e.g., in a memory of the system).

The OP System may or may not use the identifiers to verify optical prescriptions. For example, the OP System may receive (e.g., from a third party fulfillment service) patient information and/or an optical prescription associated with an optical product order for a first patient. The OP System may retrieve the stored optical prescription information and/or other information (e.g., patient information, subset of product information, etc.) and compare the retrieved information to the information received from the third party fulfillment service. If the compared information or specified portion thereof (e.g., required optical prescription information) is the same, the prescription may be verified. In some implementations, the ordered product ordered may be identified by the Third party fulfillment service and this information may be transmitted to the OP System. The OP System may determine if the ordered optical product is in the set of optical products associated with the optical prescription to determine if the prescription is valid.

In some implementations, the OP System may utilize the identifier to verify optical prescriptions and/or transmit pre-verified optical prescriptions to the third party optical product fulfillment service. For example, a patient may be transmitted an associated identifier. The patient may provide the associated identifier to the third party fulfillment service, which in turn provides the identifier to the OP System. The OP System determines if the provided identifier is in the set of identifiers stored and/or valid in the OP system. If the provided identifier is in the set of identifiers stored in the OP System, then the associated previously valid optical prescription may be retrieved. This pre-validated optical prescription may then be transmitted to the third party fulfillment service for use in filling optical product requests from the third party.

In some implementations, an identifier may be automatically removed at an expiration date of the optical prescription associated with the identifier. In some implementations, expiration date for one or more of the optical prescriptions may be determined by the OP System (e.g., based on doctor preferences, industry standards, regulations, etc.). An identifier (e.g., unique identifier) may be automatically removed when later extracted patient information indicates that the optical prescription associated with the identifiers is not valid (e.g., the optical prescription has been replaced, the optical prescription was ill suited for the patient, etc.).

A notification may be transmited to a patient associated with an identifier and/or the Third Party Fulfillment Service, if a determination is made that the first identifier is not valid. An optical prescription or portions thereof may be automatically adjusted if a determination is made that the optical prescription (e.g., as retrieved) is not valid. The optical prescription or portions thereof may be adjusted based on doctor preferences and/or retrieved optical product information. The adjusted optical prescription may be valid.

In some implementations, a patient may be offered an optical product that matches the patient's optical needs. A first patient may be identified, who visited a prescriber within a predetermined period of time, from the retrieved patient information associated with the first patient. A determination may be made whether the first patient purchased a first optical product based on the patient information associated with the first patient retrieved from the third party practice management system, based on previous optical prescription validation requests, and/or based on previously provided preverified optical prescriptions. If a determination is made that the first patient did not purchase the first optical product, a sales offer for the first optical product may be transmitted to the first patient with the preverified optical prescription associated with the first patient.

In some implementations, automatically verifying one of the optical prescriptions may include receiving a first unique identifier from the Third Party Fulfillment Services and determining whether the first unique identifier is the same as one of the stored assigned unique identifiers. If a determination is made that the first unique identifier is the same as one of the stored assigned unique identifiers, the optical prescription associated with the first unique identifier may be retrieved (e.g., from a memory of the OP System). The retrieved optical prescription may be transmitted to the Third Party Fulfillment Service as a preverified optical prescription.

In some implementations, a set of optical products that may be purchased by a patient with a specific optical prescription may be identified and utilized by the OP system. For example, a set of optical products that can be validly filled using the optical prescription information of an optical prescriptions may be identified based on doctor based preferences and/or regulations (e.g., substantially similar products identified by the doctor, the manufacturer, industry, and/or the government). The identifier information may include the set of optical products associated with an optical prescription in some implementations and/or the determined set(s) of optical products may be stored in association with the associated one or more unique identifiers.

In some implementations, automatically verifying one of the optical prescriptions may include receiving a first identifier and an identification of a first optical product from the Third Party Fulfillment Services, and determining whether the first unique identifier is the same as one of the stored assigned unique identifiers. If a determination is made that the first unique identifier is the same as one of the stored assigned unique identifiers, a determination may be made whether first optical product is in the stored set of optical products associated with the first unique identifier. A notification may be transmitted to the Third Party Fulfillment Service that the first optical product is verified if the determination is made that the first optical products is in the stored set of optical products and the optical prescription is validated.

In some implementations, an optical prescription or portion thereof may be automatically adjusted by the OP System, if a determination is made that the optical prescription is not valid as retrieved. The optical prescription or portion thereof may be adjusted based on doctor preferences and/or retrieved optical product information. The adjustment of the optical prescription may cause the adjusted optical prescription to become valid (e.g., fillable by a retailer).

In various implementations, the OP System may allow for instant and/or nearly instant verification by an optical product retailer (e.g., at the point of sale). The OP System may lower costs for verification by an optical product retailer (e.g., current costs are approximately $5/order and thus even when expensive contact lenses are ordered, costs for verification multiplies and decreases profitability over hundreds of thousands of verifications). Use of the OP system may reduce and/or eliminate time and/or costs incurred by providers (e.g., eye care professional offices) to manually verify prescriptions, which may cost ˜$10K annually per practice. In some implementations, use of the OP system may allows immediate shipping of optical products, rather than waiting the usual time period (e.g., until verified by a provider or 8 business hours, whichever is first). Use of the OP system may allow one click ordering. For example, a user may be provided with a sales offer linked to an identifier and/or a preverified optical prescription, and accepting the offer (e.g., clicking the link, texting a code to a number, etc.) may provide one step order and optical prescription verification.

In various implementations, use of an automatic prescription verification and/or preverified optical prescriptions of the OP System may reduce product errors, which may increases patient eye health. Since currently humans enter and/or verify optical prescriptions, optical prescriptions may be subject to inadvertent errors such as translation errors, misentered information, etc. Errors may be caught by the OP System quickly (e.g., when determining if an optical prescription is valid and/or adjusting an optical prescription) which may increase user satisfaction and/or increase patient eye health (e.g., since mistakes may be quickly caught and/or inhibited). Currently, over 50% of prescriptions are not being verified or incorrectly, thus, the use of the OP system may represent a vast increase in accuracy and/or patient health.

In various implementations, patient satisfaction may be increased since patients receive ordered products faster due to the OP system use of automatic verification and/or preverified optical prescriptions.

In some implementations, patients may be incentivized to utilize identifiers to provide preverified optical prescriptions. For example, an optical product retailer and/or a prescriber may pass along cost savings.

In various implementations, the OP System may provide a fast solution for doctor's offices and individuals. For example, the OP System may be capable of extracting information and transmitting notifications to users within a predetermined time period (e.g. default and/or customize by individual and/or doctor). For example, an individual may receive a fulfillment notification or request related to determining if the individual would like to fulfill a optical prescription within an hour (e.g., 10 minutes) of exiting a doctor's office. In some implementations, verifications of optical prescriptions for third party services may be provided almost immediately and/or in a short period of time (e.g., 30 minutes).

### EXAMPLES

### Example 1

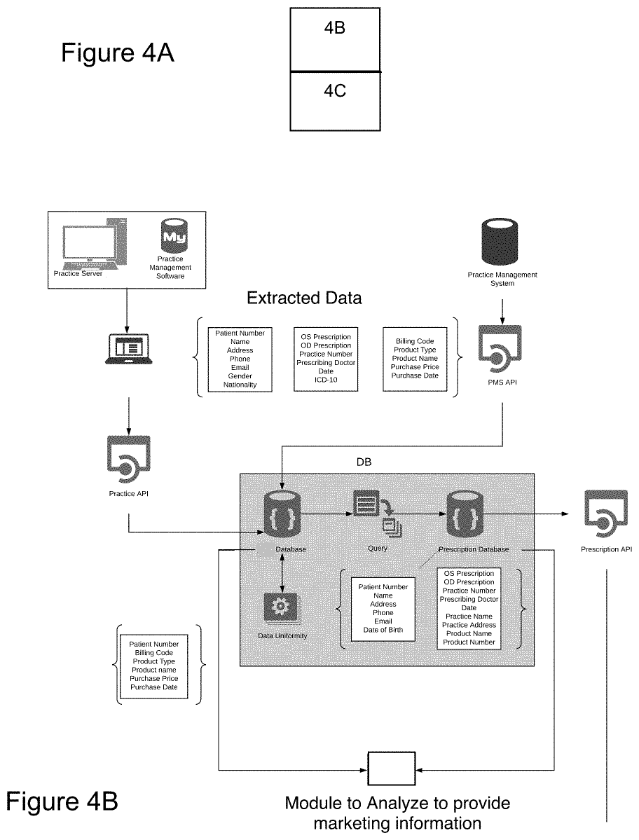

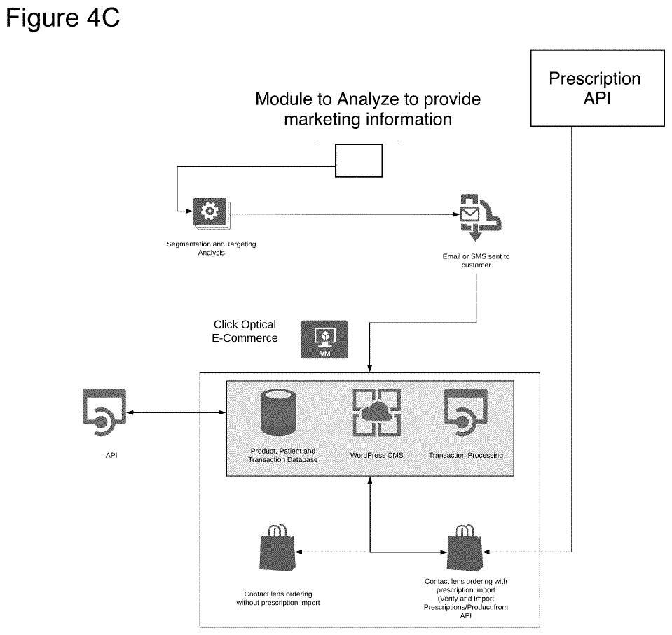

FIGS. 4A, 4B, and 4C illustrate an implementation of a process to manage fulfillment and/or prescriptions. As illustrated, data is acquired and/or generated in a Doctor's Office and Records Management Systems, such as Practice management system, may be utilized to record and/or store the data. This data may then be extracted by the OP System (e.g., an OP API, such as a practice management (PM) API may be utilized). The extracted data may include any appropriate information stored by the Records Management System, such as Patient information (e.g., identifying number; name; contact information such as address, phone, email, messaging number, contact preferences); optical prescription information (e.g., OS prescription, OD prescription, Practice Number, Prescribing Doctor, Date); billing codes and/or diagnostic codes (e.g., ICD-10); prescribing information such as product type, fulfillment information such as purchase price, quantity, and/or date; and/or other appropriate information. This extracted data or portions thereof may be stored by the OP System. The OP System may store the extracted data, supplemented extracted data (e.g., perfected optical prescriptions), and/or portions thereof such that the data may be queried, reported, extracted, presented, etc. The stored information may be analyzed to provide marketing information that may be used, in part, to transmit notifications to individuals based on the analysis such as advertisements, offers, discounts, etc. The OP System may also be utilized to fulfill optical prescriptions; remind individuals to fulfill optical prescriptions; monitor fulfillment of optical prescriptions and/or related products and/or services; and/or allow fulfillment to optical prescriptions (e.g., by verifying optical prescriptions).

### Example 2

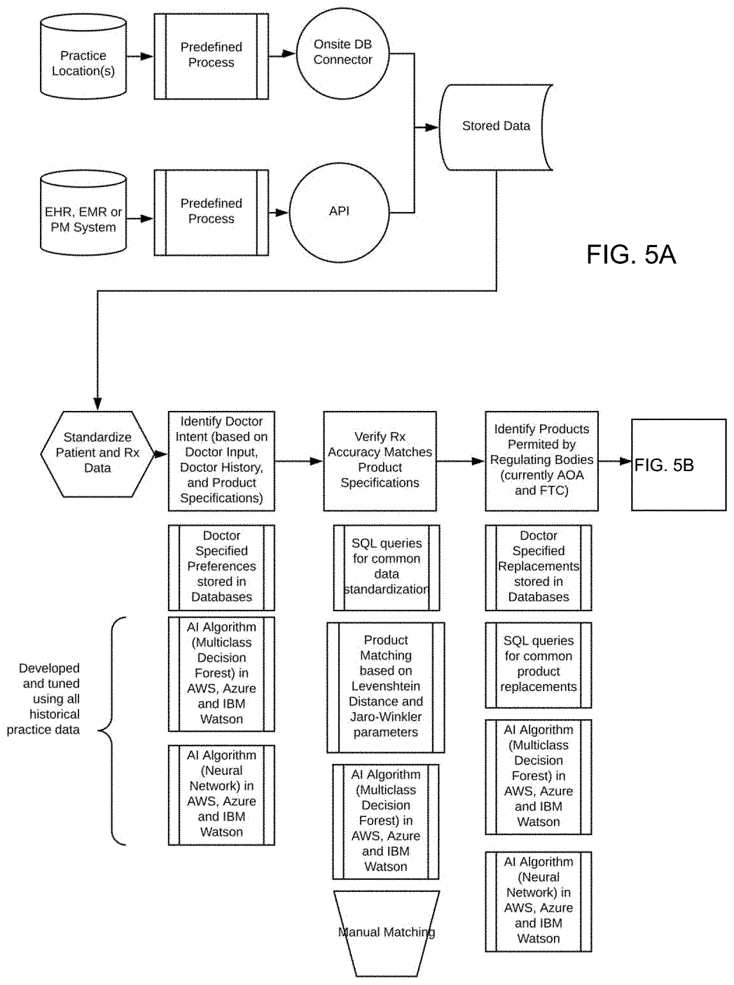

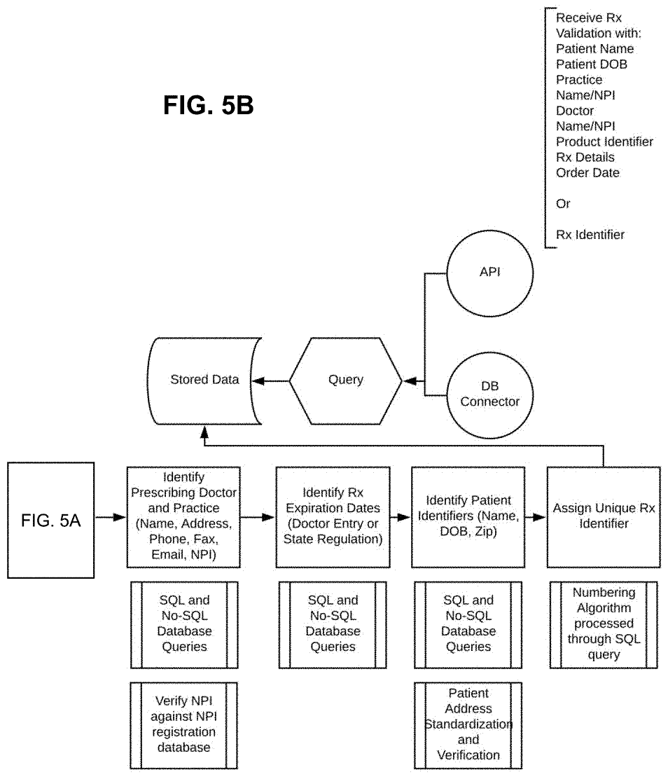

FIGS. 5A and 5B illustrate an implementation of an example process implemented by the OP System as illustrated in FIGS. 3A-3B and/or other OP Systems described herein.

### End of Examples

Although users have been described as a human, a user may be a person, a group of people, a person or persons interacting with one or more computers, and/or a computer system. Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

In various implementations, although a doctor and/or doctor's office is referenced to provide an example implementation, the OP System and processes performed by the OP System may be applicable to other prescribers (e.g., optometrists). For example, information may be retrieved by the OP API from practice management system accessible at an optometrist's office.

In various implementations, module(s) of the OP system may perform one or more of the described operations.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

The OP system may include computer programs stored at least partially on a memory of the OP System and executed at least partially by a processor of the OP system. These computer programs may also and/or alternatively be stored at least partially on and/or be executed at least partially by devices associated with individuals (e.g., user devices) and/or doctor's offices. These computer programs (also known as programs, software, software applications or code) include machine instructions for a programmable processor, and can be implemented in a high-level procedural and/or object-oriented programming language, and/or in assembly/machine language. As used herein, the term “machine-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user (e.g., doctor's office personnel and/or individuals), the systems and techniques described here can be implemented on a computer having a display device (e.g., LCD (liquid crystal display) monitor)) for displaying information to the user and a keyboard and a pointing device (e.g., a mouse or a track pad) by which the user can provide input to the computer. Other kinds of devices can be used to provide for interaction with a user as well; for example, feedback provided to the user by an output device can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), and the Internet.

The computing system may include clients and servers. A client and server are generally remote from each other and typically interact through a communication network. The relationship of client and server arises by virtue of computer programs running on the respective computers and having a client-server relationship to each other. Updates to the OP system may be pushed to clients via a network.

In various implementations, graphical user interface (GUI) may be generated by module(s) (e.g., of the OP system) and may be displayed on a presentation interface (e.g., of a user device for users such as patients, third party seller, provider, etc.). GUI may be operable to allow the user of a user device to interact with repositories and/or various interface(s). Generally, GUI provides a user with an efficient and user-friendly presentation of data provided by the system. GUI includes a plurality of displays having interactive fields, such as image icons, pull-down lists, fillable fields, and editable text operated by the user. And in one example, GUI presents an explore-type interface and receives commands from the user. It should be understood that the term graphical user interface may be used in the singular or in the plural to describe one or more graphical user interfaces in each of the displays of a particular graphical user interface. Further, GUI contemplates any graphical user interface, such as a generic web browser, that processes information in the system and/or user device and efficiently presents the information to the user. In some implementations, GUI may present a web page embedding content. The server can accept data from a user device(s) via the web browser (e.g., Microsoft Internet Explorer, Safari, or FireFox) and return the appropriate Hyper Text Markup Language (HTML) or eXtensible Markup Language (XML) responses.

Various described processes may be implemented by various systems, such as the described system(s). In addition, various operations may be added, deleted, and/or modified. In some implementations, a described process may be performed in combination with other described processes or portions thereof.

Although users have been described as a human, a user may be a person, a group of people, a person or persons interacting with one or more computers, and/or a computer system. For example, several operations and/or system components are described in terms of a single individual; however, the operations and/or system components may be utilized by a plurality of individuals (e.g., patients of a practice, members of a group such as insurance, trade association, business, warehouse club, etc.). Some operations and/or system components are described in terms of a single Doctor's Office; however, the operations and/or system components may be utilized by a plurality of Doctor's Offices and/or a plurality of Doctors or personnel associate with a Doctor's Office.

Although various optical prescriptions refer specifically to glasses and/or contact lenses, various operations and/or system components may include optical prescriptions for contact lenses and/or glasses. For example, an optical prescription may be described as being extracted for glasses but optical prescriptions may be extracted for contact lenses in addition to and/or in place of an optical prescription for glasses.

It is to be understood the implementations are not limited to particular systems or processes described which may, of course, vary. It is also to be understood that the terminology used herein is for the purpose of describing particular implementations only, and is not intended to be limiting. As used in this specification, the singular forms “a”, “an” and “the” include plural referents unless the content clearly indicates otherwise. Thus, for example, reference to “optical prescription” includes a combination of two or more optical prescriptions and reference to “contact lenses” includes different types and/or combinations of contact lenses.

Although the present disclosure has been described in detail, it should be understood that various changes, substitutions and alterations may be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method of verifying optical prescriptions for fulfillment of a plurality of optical prescriptions, the method comprising:
retrieving, for a plurality of patients, a plurality of patient information and associated optical prescriptions information from a third party patient management system;
determining if the optical prescription information for each of the associated optical prescriptions is valid as retrieved;
assigning a unique identifier for each of the optical prescriptions, wherein each of the unique identifiers is associated with identifier information, and wherein the identifier information comprises at least a portion of the retrieved patient information and the retrieved associated optical prescription;
storing the assigned unique identifiers and the associated identifier information; and
automatically transmitting a first prevalidated optical prescription in response to receiving, from a Third Party Fulfillment Service, a valid first identifier, wherein prior to transmitting a first prevalidated optical prescription:
a determination is made whether the first identifier is valid by determining whether the first identifier comprises one of the stored assigned unique identifiers; and
the first prevalidated optical prescription is retrieved based on the first identifier and associated identifier information.

2. The method of claim 1 further comprising automatically removing each of the assigned unique identifier at an expiration date of the optical prescription associated with each of the unique identifiers.

3. The method of claim 1 further comprising transmitting a notification to at least one of the patients associated with the first identifier or the Third Party Fulfillment Service if a determination is made that the first identifier is not valid.

4. The method of claim 1 further comprising:
automatically adjusting at least a part of one of the optical prescriptions if a determination is made that the optical prescription is not valid as retrieved based on at least one of doctor preferences or retrieved optical product information;
wherein the adjusted optical prescription is valid.

5. The method of claim 1 further comprising:
identifying a first patient of the plurality of the patients that visited a prescriber within a predetermined period of time from the retrieved patient information associated with the first patient;
determining whether the first patient purchased a first optical product based on the patient information associated with the first patient retrieved from the third party practice management system; and
if a determination is made that the first patient did not purchase the first optical product, transmitting a sales offer for the first optical product with the preverified optical prescription associated with the first patient.

6. The method of claim 1 further comprising transmitting a notification to one or more of the patients of the plurality of patients that includes at least one of the unique identifiers associated with each of the one or more of the patients.

7. A method of verifying optical prescriptions for fulfillment of a plurality of optical prescriptions, the method comprising:
retrieving, for a plurality of patients, a plurality of patient information and associated optical prescriptions information from a third party patient management system;
determining if optical prescription information in each of the associated optical prescriptions is valid as retrieved;
assigning a unique identifier to each of the valid optical prescriptions, wherein each of the unique identifiers is associated with identifier information, and wherein the identifier information comprises at least a portion of the retrieved patient information and the retrieved associated optical prescription;
storing the assigned unique identifiers and the associated identifier information;
automatically removing each of the assigned unique identifier at an expiration date of the optical prescription associated with each of the unique identifiers; and
automatically verifying one of the optical prescriptions associated with one of the patients based on a request from a Third Party Fulfillment Services for verification, when a first unique identifier is received from the Third Party Fulfillment Service, wherein the first unique identifier is the same as one of the stored assigned unique identifiers and is associated with the one of the optical prescriptions.

8. The method of claim 7 wherein automatically verifying one of the optical prescriptions comprises:
receiving the first unique identifier from the Third Party Fulfillment Services;
determining whether the first unique identifier is the same as one of the stored assigned unique identifiers;
if a determination is made that the first unique identifier is the same as one of the stored assigned unique identifiers, retrieving the optical prescription associated with the first unique identifier;
and transmitting the retrieved optical prescription to the Third Party Fulfillment Service as a preverified optical prescription.

9. The method of claim 7 further comprising automatically removing one or more of the assigned unique identifiers when later extracted patient information indicates that the one or more optical prescriptions associated with the one or more assigned unique identifiers is not valid.

10. The method of claim 7 further comprising for at least one of the optical prescriptions that is determined to be valid:
determining one or more sets of optical products that can be validly filled using the optical prescription information of the at least one of the optical prescriptions based on at least one of doctor based preferences or regulations, wherein the identifier information for at least one of the assigned unique identifiers further comprises one of the sets of optical products associated with the one of the optical prescriptions associated with the assigned unique identifier; and
storing the one or more determined sets of optical products in association with the associated one or more unique identifiers.

11. The method of claim 10 wherein automatically verifying one of the optical prescriptions further comprises:
receiving the first identifier and an identification of a first optical product from the Third Party Fulfillment Services;
determining whether the first unique identifier is the same as one of the stored assigned unique identifiers;
if a determination is made that the first unique identifier is the same as one of the stored assigned unique identifiers, determining whether first optical product is in the stored set of optical products associated with the first unique identifier;
and transmitting a notification to the Third Party Fulfillment Service that the first optical product is verified if the determination is made that the first optical products is in the stored set of optical products and the optical prescription is validated.

12. The method of claim 7 further comprising determining an expiration date for one or more of the optical prescriptions.

13. The method of claim 7 further comprising transmitting a notification to a first patient of the plurality of patients that includes a first unique identifier of the unique identifiers that is associated with the first patient.

14. The method of claim 10 further comprising:
determining whether a first patient of the plurality of patients purchased a first optical product based on the patient information retrieved from the third party practice management system, wherein the first optical product is in the set of optical products associated with one of the unique identifiers of the first patient; and
if a determination is made that the first patient did not purchase the first optical product, transmitting a sales offer with preverified optical prescription information for the first optical product.

15. The method of claim 10 further comprising:
identifying a first patient of the plurality of patients that visited a doctor within a predetermined period of time from the retrieved patient information;
determining a first optical product in the set of optical products associated with one of the unique identifiers of the first patient;
determining whether the first patient purchased the first optical product based on the patient information retrieved from at least one of the third party practice management system and or previously issued optical prescription verifications; and
if a determination is made that the individual did not purchase the first optical product, transmitting a preverified sales offer for the first optical product to the first patient.

16. The method of claim 7 further comprising:
automatically adjusting at least a portion of one or more of the optical prescriptions if a determination is made that the one or more optical prescriptions is not valid as retrieved, wherein the one or more optical prescriptions is adjusted based on at least one of doctor preferences or retrieved optical product information;
wherein the one or more adjusted optical prescriptions are valid.

17. A system for verifying optical prescriptions, the system comprising:
a memory storing:
retrieved patient information for a plurality of patients;
one or more optical prescriptions for one or more of the patients;
a plurality of identifiers, wherein each identifier is associated with one of the optical prescriptions;
associations between at least one of the retrieved patient information, the plurality of patients, the one or more optical prescriptions, and the plurality of identifiers; and
instructions for obtaining and verifying optical prescriptions;
one or more processors configured to execute one or more of the instructions to:
retrieve, for the plurality of patients, the patient information and the associated optical prescriptions information from a third party patient management system;
determine if at least a portion of each of the optical prescriptions is valid as retrieved;
assign a unique identifier for each of the optical prescriptions, wherein each of the unique identifiers is associated with identifier information, and wherein the identifier information comprises at least a portion of the retrieved patient information and the retrieved associated optical prescription;
store the assigned unique identifiers and the associated identifier information in the memory; and
automatically transmit a first prevalidated optical prescription in response to receiving, from a Third Party Fulfillment Service, a valid first identifier, wherein prior to transmitting a first prevalidated optical prescription:
a determination is made whether the first identifier is valid by determining whether the first identifier comprises one of the stored assigned unique identifiers; and
the first prevalidated optical prescription is retrieved based on the first identifier and associated identifier information.

18. The system of claim 17 wherein the one or more processors are further configured to execute one or more of the instructions to automatically remove each of the assigned unique identifier at the expiration date of the optical prescription associated with each of the unique identifiers.

19. The system of claim 17 wherein the one or more processors are further configured to execute one or more of the instructions to:
automatically adjust at least a portion of the optical prescriptions if a determination is made that the optical prescription is not valid as retrieved based on at least one of doctor preferences or retrieved optical product information, wherein the adjusted at least a portion of the optical prescriptions becomes valid based on the adjustment.

20. The system of claim 17 wherein the one or more processors are further configured to execute one or more of the instructions to transmit a notification to one of the patients of the plurality of patients that includes one of the unique identifiers associated with the patient.
